(19) United States
(12) Patent Application Publication
(10) Pub. No.: US 2026/0092335 A1
(43) Pub. Date: Apr. 2, 2026

(54) VIRUS NUCLEIC ACID SAMPLE DILUENT, VIRUS NUCLEIC ACID SAMPLE EXTRACTION KIT, AND VIRUS NUCLEIC ACID EXTRACTION METHOD

(71) Applicant: SHANGHAI BIOGERM MEDICAL TECHNOLOGY CO., LTD., Shanghai (CN)

(72) Inventors: Ziming SHEN, Shanghai (CN); Baihui ZHAO, Shanghai (CN); Chunyan LI, Shanghai (CN); Chunlei ZHANG, Shanghai (CN)

(21) Appl. No.: 19/113,562

(22) PCT Filed: Dec. 30, 2022
(86) PCT No.: PCT/CN2022/144219

§ 371 Date: Mar. 20, 2025

(30) Foreign Application Priority Data
Sep. 27, 2022 (CN) 202211182551.5

(51) Int. Cl. C12Q 1/70; C12N 15/10; C12Q 1/6806

(52) U.S. Cl. CPC C12Q 1/70; C12N 15/1003; C12Q 1/6806

(57) ABSTRACT

A virus nucleic acid sample diluent, sample extraction kit, and extraction method. A release agent compatible with various sampling sets. Critical micelle concentration of a used surfactant is reduced by adjusting types and proportions of components. The lysis of a cell and a pathogen can be ensured, and the inhibitory effect on subsequent detection systems can be reduced. Under the condition that an ion exchange resin is added, a protein and metal ions (e.g., calcium and magnesium ions) that affect a subsequent reaction can be adsorbed from a sample, and cell and virus lysis can also be promoted, for enhanced compatibility of the system. Compatible with various Quantitative polymerase chain reaction (qPCR) detection systems at the same time, and can also be used for an isothermal recombinase-aided amplification (RAA) amplification system. Suitable for a liquid detection system and a dry powder detection system with a large sample volume.

### CROSS-REFERENCE TO RELATED APPLICATION(S)

The present disclosure claims priority to the Chinese Patent Application No. CN202211182551.5 entitled “VIRUS NUCLEIC ACID SAMPLE DILUENT, VIRUS NUCLEIC ACID SAMPLE EXTRACTION KIT, AND VIRUS NUCLEIC ACID EXTRACTION METHOD” and filed with China National Intellectual Property Administration on Sep. 27, 2022, which is incorporated herein by reference in its entirety.

### TECHNICAL FIELD

The present disclosure relates to the technical field of molecular detection, in particular to a viral nucleic acid sample diluent, kit and method for extracting viral nucleic acid from samples.

### BACKGROUND

With the spread of the COVID-19 pandemic, molecular detection technologies have undergone rapid development. Molecular diagnostic technology is a method that detects pathogens based on their nucleic acids by identifying specific nucleic acid sequences of the pathogens. Therefore, obtaining nucleic acids suitable for the reaction is crucial for the accurate detection of pathogens, and the speed of nucleic acid extraction is a bottleneck that limits the detection speed. Conventional nucleic acid extraction methods, such as centrifugal column or magnetic bead-based techniques, not only require specialized equipment like centrifuges and magnetic racks but also involve relatively complex procedures, including lysis, washing, and elution steps.

As detection demands continue to increase, the need for home self-testing and rapid detection has gradually gained attention. To meet such demands, it is necessary to develop nucleic acid extraction methods that do not rely on specialized instruments while maintaining sufficient detection capabilities. However, existing nucleic acid release agents on the market often fail to achieve satisfactory results after releasing nucleic acids. Moreover, most nucleic acid release agents are designed for only one specific detection method and cannot be effectively used in isothermal detection systems that allow for rapid amplification.

### SUMMARY

The present disclosure provides a viral nucleic acid sample diluent comprising an anionic surfactant, sodium hydroxide, EDTA, trehalose, and an ion exchange resin. An addition amount of the EDTA is 0-3% according to mass-volume ratio; an addition amount of the anionic surfactant is 0.010%-0.050%; and an addition amount of the ion exchange resin is 0-5%. Optionally, the addition amount of EDTA is 1%. Optionally, the addition amount of the anionic surfactant is 0.02%. Optionally, the addition amount of the ion exchange resin is 2.5%.

Optionally, the anionic surfactant is selected from sodium dodecyl sulfate, sodium tetradecyl sulfate, sodium hexadecyl sulfate, and sodium octadecyl sulfate. Optionally, the anionic surfactant is sodium hexadecyl sulfate.

Optionally, a concentration of the sodium hydroxide is 0.16-1.28 mmol/L.

Optionally, the concentration of the sodium hydroxide is 0.32 mmol/L.

Optionally, the viral nucleic acid sample diluent also comprises one or a combination of two or more of polyol, sodium chloride, or NP-40.

Optionally, the polyol is selected from ethylene glycol and propylene glycol.

Optionally, the polyol is 0-10% propylene glycol according to mass-volume ratio. Optionally, the polyol is 5% propylene glycol according to mass-volume ratio.

Optionally, a concentration of the NP-40 is 1%-5% according to mass-volume ratio. Optionally, the concentration of the NP-40 is 1% according to mass-volume ratio.

Optionally, a concentration of the sodium chloride is 50-150 mmol/L. Optionally, the concentration of the sodium chloride is 100 mmol/L.

Optionally, the ion exchange resin is selected from chelex and bio-rex 70, and the chelex has a specification of 50-400 mesh.

Optionally, the ion exchange resin is bio-rex 70.

Optionally, a concentration of the trehalose is 0.05-0.1 mmol/L. Optionally, the concentration of the trehalose is 0.075 mmol/L.

The present disclosure also provides a kit for extracting viral nucleic acid from samples, comprising the viral nucleic acid sample diluent according to any one of the above.

Optionally, the kit for extracting viral nucleic acid from samples also comprises a heating device.

The present disclosure also provides a method for extracting viral nucleic acid from samples, comprising: uniformly mixing a virus sample with the viral nucleic acid sample diluent according to any one of the above, and obtaining the viral nucleic acid after completing the reaction.

Optionally, the reaction is performed for a period of 5-15 min.

Optionally, the method for extracting viral nucleic acid from samples comprises the following steps: uniformly mixing the virus sample with the viral nucleic acid sample diluent according to any one of the above and then heating, and obtaining the viral nucleic acid after completing the reaction.

Optionally, the heating is performed at a temperature of 90-110° C., with a period of 1-5 min.

The present disclosure also provides use of the viral nucleic acid sample diluent or the kit for extracting viral nucleic acid from samples according to any one of the above in the diagnosis of a virus infection.

Optionally, the virus comprises a novel coronavirus.

The present disclosure also provides a method for diagnosing a disease associated with a virus infection in a subject, comprising:
A) uniformly mixing the viral nucleic acid sample diluent or a reagent in the kit for extracting viral nucleic acid from samples according to any one of the above with a virus sample from the subject, obtaining a viral nucleic acid after completing the reaction, and performing a detection via a virus detection reagent; and
B) determining the source of the virus.

Optionally, the virus comprises a novel coronavirus.

### DETAILED DESCRIPTION

In order to make the objectives, technical solutions and advantages of the embodiments of the present disclosure clearer, the technical solutions in the embodiments of the present disclosure will be clearly and completely described below, and it is obvious that the described embodiments are some but not all embodiments of the present disclosure. Therefore, based on the embodiments of the present disclosure, all other embodiments obtained by those of ordinary skill in the art without creative efforts shall fall within the claimed scope of the present disclosure.

One embodiment of the present disclosure provides a viral nucleic acid sample diluent comprising an anionic surfactant, sodium hydroxide, EDTA, trehalose, and an ion exchange resin. Alternatively, the sodium hydroxide in the viral nucleic acid sample diluent may also be replaced by any basic reagent comprising a hydroxyl ion. Optionally, the basic reagent may comprise, but is not limited to, at least one of potassium hydroxide, lithium hydroxide, and ammonium hydroxide.

Optionally, the addition amount of EDTA is 0-3% according to mass-volume ratio, comprising but not limited to, for example, 0.4-2.8%, 0.8-2.5%, or 1.2-2.2%, such as 0.5%, 1%, 1.5%, 2%, 2.5%, or 3%, or a value in the interval between any two endpoints, optionally 1%. The addition amount of the anionic surfactant is 0.010%-0.050%, comprising but not limited to, for example, 0.014%-0.045%, 0.020%-0.040%, or 0.025%-0.035%, such as 0.010%, 0.015%, 0.02%, 0.025%, 0.03%, 0.035%, 0.04%, 0.045%, or 0.050%, or a value in the interval between any two endpoints, optionally 0.02%. The addition amount of the ion exchange resin is 0-5%, comprising but not limited to, for example, 0.5-4.5%, 1.0-4.0%, or 2.5-3.5%, such as 0.5%, 1%, 1.5%, 2%, 2.5%, 3%, 3.5%, 4%, 4.5%, or 5%, or a value in the interval between any two endpoints, optionally 2.5%.

Most nucleic acid release agents available on the market are not suitable for use in isothermal amplification or dry powder detection systems due to the lytic components in their formulations. This product effectively addresses this issue by reducing the critical micelle concentration of the lytic components. It demonstrates excellent performance in both RAA and qPCR detection methods. Furthermore, it can be applied in dry powder detection systems for the aforementioned techniques, thus enhancing the detection sensitivity.

The EDTA in the present disclosure, as a commonly used chelating agent for metal ions, is primarily used to mitigate inhibitors and metal ions that may be present in the sample and various sampling kits. It has stable physicochemical properties and does not introduce any specific metal ions except for sodium ions. However, an excessively high concentration of EDTA can chelate magnesium ions required for subsequent amplification reactions, leading to reaction inhibition.

The ion exchange resin in the present disclosure mainly functions to assist in the lysis of cells and viruses and to adsorb impurities.

In an optional embodiment, the anionic surfactant is selected from sodium dodecyl sulfate, sodium tetradecyl sulfate, sodium hexadecyl sulfate, and sodium octadecyl sulfate. In an optional embodiment, the anionic surfactant is selected from at least one of sodium dodecyl sulfate, sodium tetradecyl sulfate, sodium hexadecyl sulfate, and sodium octadecyl sulfate. Optionally, the anionic surfactant is sodium hexadecyl sulfate.

The reason for selecting sodium hexadecyl sulfate in the present disclosure is: according to the properties of surfactants, the same series of surfactants with the same hydrophilic group has lower critical micelle concentration if the lipophilic group is larger. Additionally, from an economic perspective, sodium hexadecyl sulfate is a cost-effective choice.

In an optional embodiment, the concentration of sodium hydroxide is 0.16-1.28 mmol/L, comprising but not limited to, for example, 0.20-1.20 mmol/L, 0.40-1.0 mmol/L, or 0.60-0.80 mmol/L, such as 0.16 mmol/L, 0.24 mmol/L, 0.32 mmol/L, 0.40 mmol/L, 0.48 mmol/L, 0.56 mmol/L, 0.64 mmol/L, 0.72 mmol/L, 0.80 mmol/L, 0.88 mmol/L, 0.96 mmol/L, 1.04 mmol/L, 1.12 mmol/L, 1.20 mmol/L, or 1.28 mmol/L, or a value in the interval between any two endpoints, optionally 0.32 mmol/L.

In the present disclosure, the alkaline environment is primarily provided by NaOH. To minimize inhibition of the detection system, the concentration of NaOH is maintained between 0.16 and 1.28 mmol/L, and an optimal effect is achieved at a concentration of 0.32 mmol/L.

In an optional embodiment, also comprised is one or a combination of two or more of polyol, sodium chloride, or NP-40.

In order to further reduce the concentration of the anionic surfactant and improve the compatibility of the system, at least one of polyol, sodium chloride, or NP-40 is added and compounded with sodium hexadecyl sulfate in the present disclosure, so that the critical micelle concentration is further reduced.

In an optional embodiment, the polyol is selected from ethylene glycol and propylene glycol. Optionally, the polyol is selected from at least one of ethylene glycol and propylene glycol. These two polyols have strong polarity, which allows them to engage in intense competitive binding with water molecules, thereby reducing the critical micelle concentration of the surfactant.

Optionally, the polyol is 0-10% propylene glycol according to mass-volume ratio, comprising but not limited to, for example, 0.1-9%, 1.5-8%, or 2.5-7%, such as 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%, or a value in the interval between any two endpoints; optionally, the polyol is 5% propylene glycol.

Optionally, the concentration of NP-40 is 1%-5% according to mass-volume ratio, comprising but not limited to, for example, 1.4%-4.5%, 2.0%-4.0%, or 2.5%-3.5%, such as 1%, 1.5%, 2%, 2.5%, 3%, 3.5%, 4%, 4.5%, or 5%, or a value in the interval between any two endpoints, optionally 1%;

optionally, the concentration of sodium chloride is 50-150 mmol/L, comprising but not limited to, for example, 60-140 mmol/L, 70-120 mmol/L, or 80-110 mmol/L, such as 50 mmol/L, 55 mmol/L, 60 mmol/L, 65 mmol/L, 70 mmol/L, 75 mmol/L, 80 mmol/L, 85 mmol/L, 90 mmol/L, 95 mmol/L, 100 mmol/L, 105 mmol/L, 110 mmol/L, 115 mmol/L, 120 mmol/L, 125 mmol/L, 130 mmol/L, 135 mmol/L, 140 mmol/L, 145 mmol/L, or 150 mmol/L, or a value in the interval between any two endpoints, optionally 100 mmol/L.

In the present disclosure, NP-40 and sodium chloride are both a compound of surfactants, and the two substances have small adverse effect on the subsequent detection reaction.

In an optional embodiment, the ion exchange resin is selected from chelex or bio-rex 70, optionally bio-rex 70.

The chelex has a specification of 50-400 mesh, comprising but not limited to, for example, 80-380 mesh, 100-350 mesh, or 150-300 mesh, such as 50 mesh, 55 mesh, 60 mesh, 65 mesh, 70 mesh, 75 mesh, 80 mesh, 85 mesh, 90 mesh, 95 mesh, 100 mesh, 110 mesh, 120 mesh, 130 mesh, 140 mesh, 150 mesh, 160 mesh, 170 mesh, 180 mesh, 190 mesh, 200 mesh, 220 mesh, 240 mesh, 260 mesh, 280 mesh, 300 mesh, 330 mesh, 360 mesh, 390 mesh, or 400 mesh, or a value in the interval between any two endpoints.

In an optional embodiment, the concentration of trehalose is 0.05-0.1 mmol/L, comprising but not limited to, for example, 0.06-0.09 mmol/L, 0.065-0.085 mmol/L, or 0.07-0.09 mmol/L, such as 0.05 mmol/L, 0.055 mmol/L, 0.06 mmol/L, 0.065 mmol/L, 0.07 mmol/L, or 0.075 mmol/L, or a value in the interval between any two endpoints, optionally 0.075 mmol/L.

The trehalose in the present disclosure, as a commonly used PCR enhancer, is added to the system to mainly play a role in enhancing the stability of the system.

An embodiment of the present disclosure provides a kit for extracting viral nucleic acid from samples comprising the viral nucleic acid sample diluent according to any one of the aforementioned embodiments.

Optionally, the kit for extracting viral nucleic acid from samples also comprises a heating device.

An embodiment of the present disclosure provides a method for extracting viral nucleic acid from samples. A virus sample is mixed uniformly with the viral nucleic acid sample diluent according to any one of the aforementioned embodiments, and after the reaction is complete, the viral nucleic acid is obtained.

Optionally, the reaction is performed for a period of 5-15 min, comprising but not limited to, for example, 6-12 min, 7-11 min, or 8-10 min, such as 5 min, 6 min, 7 min, 8 min, 9 min, 10 min, 11 min, 12 min, 13 min, 14 min, or 15 min, or a value in the interval between any two endpoints.

In an optional embodiment, the method for extracting viral nucleic acid from samples comprises the following steps: uniformly mixing the virus sample with the viral nucleic acid sample diluent according to any one of the above and then heating, and obtaining the viral nucleic acid after completing the reaction.

Optionally, the heating is performed at a temperature of 90-110° C., comprising but not limited to, for example, 92-108° C., 94-106° C., or 96-104° C., such as 90° C., 92° C., 94° C., 96° C., 98° C., 100° C., 102° C., 104° C., 106° C., 108° C., or 110° C., or a value in the interval between any two endpoints; and the heating is performed for a period of 1-5 min, comprising but not limited to, for example, 1.5-4.5 min, 2.5-4.0 min, or 3.5-4.0 min, such as 1 min, 2 min, 3 min, 4 min, or 5 min, or a value in the interval between any two endpoints.

For example, using under room temperature: mixing the virus sample and a viral nucleic acid sample release agent according to the volume ratio of 1:1, standing for 10 min after fully mixing, and collecting the mixture for subsequent detection. In the context of the present disclosure, “room temperature” is usually referred to as the temperature range of 20° C.-35° C.

For another example, using under heating: mixing the virus sample and the viral nucleic acid release agent according to the volume ratio of 1:1, mixing uniformly, heating in a metal bath at 95° C. for 3 min, and collecting the mixture for subsequent detection.

Alternatively, using with an accompanying heating bag: in a 1.5 mL centrifuge tube, mixing the virus sample and a viral nucleic acid release agent according to the volume ratio of 1:1, mixing uniformly, adding about 5 mL of tap water or mineral water to the heating bag, heating the 1.5 mL centrifuge tube in the heating bag for 3 min, cooling the 1.5 mL EP tube slightly after the timing is complete, and collecting the mixture for subsequent experiment.

An embodiment of the present disclosure also provides use of the viral nucleic acid sample diluent or the kit for extracting viral nucleic acid from samples according to any one of the above in the diagnosis of a virus infection.

Optionally, the virus comprises a novel coronavirus.

An embodiment of the present disclosure also provides a method for diagnosing a disease associated with a virus infection in a subject, comprising:
A) uniformly mixing the viral nucleic acid sample diluent or a reagent in the kit for extracting viral nucleic acid from samples according to any one of the above with a virus sample from the subject, obtaining a viral nucleic acid after completing the reaction, and performing a detection via a virus detection reagent; and
B) determining the source of the virus.

Optionally, the virus comprises a novel coronavirus.

The present disclosure provides a more flexible viral nucleic acid release agent with more abundant usage scenarios for the deficiency of the existing nucleic acid release agent products. It is expected that the viral nucleic acid release agent can be used at room temperature, and can be used under heating without energy in combination with a suitable consumable, so as to obtain a better nucleic acid releasing effect.

The viral nucleic acid sample release agent provided by the present disclosure can be compatible with various sampling sets, and by adjusting the types and the proportions of the components, the critical micelle concentration of the used surfactant is reduced to the maximum extent, so that the lysis on cells and pathogens can be ensured, and the inhibition on the subsequent detection system can also be reduced. According to the present disclosure, when an ion exchange resin is added, proteins, metal ions such as calcium and magnesium which have influences on subsequent reactions, and the like in a sample can be adsorbed, cell and virus lysis can be promoted, and the compatibility of the system is enhanced.

According to the present disclosure, in the process of providing the viral nucleic acid sample, the viral nucleic acid can be obtained by placing the sample at room temperature for 5 min, and the viral nucleic acid can also be obtained by extracting the nucleic acid under the condition of heating at 95° C. for 3 min. The subsequent detection result of the obtained nucleic acid is close to that of the extraction by a magnetic bead-based nucleic acid extraction reagent, and the extraction method can also be directly used for processing the swab samples and then carrying out nucleic acid amplification.

The detection system of the present disclosure has relatively good compatibility, can be compatible with various qPCR detection systems, and can also be used for an isothermal RAA amplification system. The present disclosure is not only suitable for a liquid detection system, but also can be used for a dry powder detection system with a large sample size.

The present disclosure, when used in combination with self-heating consumables, can provide a relatively good nucleic acid releasing effect in a short time.

### EXAMPLES

Some examples of the present disclosure are described in detail below. The examples and features of the examples described below can be combined with each other without conflict.

### Example 1

This example provides a viral nucleic acid sample release agent comprising the following: 0.16-1.28 mmol/L NaOH, 0.5% (mass-volume ratio) sodium dodecyl sulfate, 10% (mass-volume ratio) propylene glycol, 5% (mass-volume ratio) NP-40, 150 mmol/L NaCl, 0%-3% (mass-volume ratio) EDTA, 0.05-0.1 mmol/L trehalose, and 5% (mass-volume ratio) bio-rex 70. The specific test plan is as follows in Table 1 below.

TABLE 1
Test plan

| | NaOH | EDTA | Trehalose |
|---|---|---|---|
| Formula 1 | 0.16 | 3% | 0.1 mmol/L |
| Formula 2 | 0.32 | 3% | 0.1 mmol/L |
| Formula 3 | 0.64 | 3% | 0.1 mmol/L |
| Formula 4 | 1.28 | 3% | 0.1 mmol/L |
| Formula 5 | 0.32 | 0% | 0.1 mmol/L |
| Formula 6 | 0.32 | 1% | 0.1 mmol/L |
| Formula 7 | 0.32 | 2% | 0.1 mmol/L |
| Formula 8 | 0.32 | 3% | 0.1 mmol/L |
| Formula 9 | 0.32 | 1% | 0.05 mmol/L |
| Formula 10 | 0.32 | 1% | 0.075 mmol/L |
| Formula 11 | 0.32 | 1% | 0.1 mmol/L |

### Experimental Method

### (1) Sample Preparation

The pseudovirus of novel coronavirus was taken at an initial concentration of 106 copies/mL and diluted to 105 copies/mL for later use using the sampling set from Youkanghengye after the negative swab was collected.

### (2) Sample Treatment and Detection

The prepared pseudovirus samples were separately treated at room temperature and under a heated condition using the viral nucleic acid sample release agents with different formulas in this example. The sample size was 20 μL, and the dose of the release agent was 20 μL. The room temperature treatment was that the pseudovirus sample and the viral nucleic acid sample release agent were mixed uniformly, and the mixture was left to stand at room temperature for 10 min. The heating treatment was that the pseudovirus sample and the viral nucleic acid sample release agent were mixed uniformly, and the mixture was placed in a 95° C. metal bath for 3 min. The extracted nucleic acids were detected using a commercially available novel coronaviral nucleic acid detection kit (fluorescence PCR method) (Shanghai BioGerm Medical Technology Co., Ltd.).

The detection results are shown in Table 2 and Table 3 below.

TABLE 2
Detection results of samples treated at room temperature

| Room temperature | | | | | | Repeti- | Repeti- | Mean |
|---|---|---|---|---|---|---|---|---|
| treatment | NaOH | EDTA | Trehalose | | Target | tion 1 | tion 2 | value |
| Formula 1 | 0.16 | 3% | 0.1 | mmol/L | O gene | 33.01 | 33.21 | 33.11 |
| | | | | | N gene | 34.10 | 33.94 | 34.02 |
| Formula 2 | 0.32 | 3% | 0.1 | mmol/L | O gene | 32.33 | 32.45 | 32.39 |
| | | | | | N gene | 33.21 | 33.06 | 33.14 |
| Formula 3 | 0.64 | 3% | 0.1 | mmol/L | O gene | 32.64 | 32.70 | 32.67 |
| | | | | | N gene | 33.50 | 33.77 | 33.64 |
| Formula 4 | 1.28 | 3% | 0.1 | mmol/L | O gene | 34.01 | 34.38 | 34.20 |
| | | | | | N gene | 34.78 | 34.92 | 34.85 |
| Formula 5 | 0.32 | 0% | 0.1 | mmol/L | O gene | 32.97 | 32.88 | 32.93 |
| | | | | | N gene | 33.40 | 33.76 | 33.58 |
| Formula 6 | 0.32 | 1% | 0.1 | mmol/L | O gene | 32.20 | 32.03 | 32.12 |
| | | | | | N gene | 33.05 | 33.14 | 33.10 |
| Formula 7 | 0.32 | 2% | 0.1 | mmol/L | O gene | 34.78 | 35.42 | 35.10 |
| | | | | | N gene | 36.55 | 36.61 | 36.58 |
| Formula 8 | 0.32 | 3% | 0.1 | mmol/L | O gene | 35.01 | 34.88 | 34.95 |
| | | | | | N gene | 36.97 | 37.55 | 37.26 |
| Formula 9 | 0.32 | 1% | 0.05 | mmol/L | O gene | 32.44 | 32.14 | 32.29 |
| | | | | | N gene | 33.20 | 33.38 | 33.29 |
| Formula 10 | 0.32 | 1% | 0.075 | mmol/L | O gene | 32.15 | 32.20 | 32.18 |
| | | | | | N gene | 33.01 | 33.30 | 33.16 |
| Formula 11 | 0.32 | 1% | 0.1 | mmol/L | O gene | 32.78 | 32.86 | 32.82 |
| | | | | | N gene | 33.38 | 33.65 | 33.52 |

TABLE 3
Detection results of samples heated

| Heating | NaOH | EDTA | Trehalose | | | Repetition 1 | Repetition 2 | Mean value |
|---|---|---|---|---|---|---|---|---|
| Formula 1 | 0.16 | 0.03 | 0.1 | mmol/L | O gene | 31.96 | 32.11 | 32.04 |
| | | | | | N gene | 33.05 | 32.91 | 32.98 |
| Formula 2 | 0.32 | 0.03 | 0.1 | mmol/L | O gene | 31.32 | 31.39 | 31.35 |
| | | | | | N gene | 32.13 | 32.05 | 32.09 |
| Formula 3 | 0.64 | 0.03 | 0.1 | mmol/L | O gene | 31.60 | 31.62 | 31.61 |
| | | | | | N gene | 32.44 | 32.76 | 32.60 |
| Formula 4 | 1.28 | 0.03 | 0.1 | mmol/L | O gene | 32.98 | 33.29 | 33.14 |
| | | | | | N gene | 33.69 | 33.83 | 33.76 |
| Formula 5 | 0.32 | 0 | 0.1 | mmol/L | O gene | 31.95 | 31.86 | 31.90 |
| | | | | | N gene | 32.32 | 32.75 | 32.54 |
| Formula 6 | 0.32 | 0.01 | 0.1 | mmol/L | O gene | 31.14 | 30.99 | 31.06 |
| | | | | | N gene | 32.00 | 32.11 | 32.05 |
| Formula 7 | 0.32 | 0.02 | 0.1 | mmol/L | O gene | 33.69 | 34.35 | 34.02 |
| | | | | | N gene | 35.48 | 35.53 | 35.51 |
| Formula 8 | 0.32 | 0.03 | 0.1 | mmol/L | O gene | 33.92 | 33.84 | 33.88 |
| | | | | | N gene | 35.88 | 36.46 | 36.17 |
| Formula 9 | 0.32 | 0.01 | 0.05 | mmol/L | O gene | 31.38 | 31.11 | 31.25 |
| | | | | | N gene | 32.12 | 32.29 | 32.20 |
| Formula 10 | 0.32 | 0.01 | 0.075 | mmol/L | O gene | 31.06 | 31.20 | 31.13 |
| | | | | | N gene | 31.97 | 32.26 | 32.12 |
| Formula 11 | 0.32 | 0.01 | 0.1 | mmol/L | O gene | 31.76 | 31.80 | 31.78 |
| | | | | | N gene | 32.38 | 32.63 | 32.51 |

As can be seen from the above detection results, the NaOH concentration is optionally 0.32 mmol/L, the EDTA concentration is optionally 1%, and the trehalose concentration is optionally 0.075 mmol/L.

It should be noted that, the role that EDTA plays in the system depends on the sampling tube used for sampling. If water is filled in the sampling tube or the components in the sampling tube are simple, EDTA may be not added, but EDTA needs to be added for most of the non-inactivated sampling tubes, and from the above experimental results, in the diluent provided by the present disclosure, a 1% addition amount is enough to chelate metal ions therein, and an excessive addition amount is likely to inhibit the reaction.

### Example 2

This example provides a viral nucleic acid sample release agent comprising the following: 0.32 mmol/L NaOH, 0.01-0.05% (mass/volume ratio) anionic surfactant (comprising but not limited to sodium dodecyl sulfate, sodium tetradecyl sulfate, and sodium hexadecyl sulfate), 10% (mass/volume ratio) propylene glycol, 5% (mass/volume ratio) NP-40, 150 mmol/L NaCl, 1% (mass/volume ratio) EDTA, 0.075 mmol/L trehalose, and 5% (mass/volume ratio) bio-rex 70. The specific test plan is as follows in Table 4 below.

TABLE 4
Test plan

| | Surfactant type | Concentration |
|---|---|---|
| Formula 1 | Sodium dodecyl sulfate | 0.050% |
| Formula 2 | Sodium tetradecyl sulfate | 0.050% |
| Formula 3 | Sodium hexadecyl sulfate | 0.050% |
| Formula 4 | Sodium dodecyl sulfate | 0.1% |
| Formula 5 | Sodium tetradecyl sulfate | 0.1% |
| Formula 6 | Sodium hexadecyl sulfate | 0.1% |

The experimental method was the same as that of Example 1, and the detection results are shown in Table 5 and Table 6 below.

TABLE 5
Detection results of samples treated at room temperature

| Room temperature | | | | Repeti- | Repeti- | Mean |
|---|---|---|---|---|---|---|
| treatment | Surfactant type | Concentration | | tion 1 | tion 2 | value |
| Formula 1 | Sodium dodecyl | 0.050% | O gene | 32.01 | 32.04 | 32.02 |
| | sulfate | | | | | |
| | | | N gene | 33.06 | 33.05 | 33.06 |
| Formula 2 | Sodium tetradecyl | 0.050% | O gene | 32.02 | 32.02 | 32.02 |
| | sulfate | | | | | |
| | | | N gene | 33.01 | 33.04 | 33.03 |
| Formula 3 | Sodium hexadecyl | 0.050% | O gene | 32.01 | 32.04 | 32.02 |
| | sulfate | | | | | |
| | | | N gene | 33.01 | 33.05 | 33.03 |
| Formula 4 | Sodium dodecyl | 0.1% | O gene | 34.81 | 34.24 | 34.53 |
| | sulfate | | | | | |
| | | | N gene | 35.09 | 35.67 | 35.38 |
| Formula 5 | Sodium tetradecyl | 0.1% | O gene | 34.39 | 34.89 | 34.64 |
| | sulfate | | | | | |
| | | | N gene | 35.38 | 35.62 | 35.50 |
| Formula 6 | Sodium hexadecyl | 0.1% | O gene | 34.58 | 34.76 | 34.67 |
| | sulfate | | | | | |
| | | | N gene | 35.98 | 35.67 | 35.83 |

TABLE 6
Detection results of samples heated

| Heating | Surfactant | Concen- | | Repeti- | Repeti- | Mean |
|---|---|---|---|---|---|---|
| treatment | type | tration | | tion 1 | tion 2 | value |
| Formula 1 | Sodium | 0.050% | O | 31.06 | 31.08 | 31.07 |
| | dodecyl | | gene | | | |
| | sulfate | | N | 32.09 | 32.09 | 32.09 |
| | | | gene | | | |
| Formula 2 | Sodium | 0.050% | O | 31.08 | 31.10 | 31.09 |
| | tetradecyl | | gene | | | |
| | sulfate | | N | 32.07 | 32.09 | 32.08 |
| | | | gene | | | |
| Formula 3 | Sodium | 0.050% | O | 31.05 | 31.08 | 31.07 |
| | hexadecyl | | gene | | | |
| | sulfate | | N | 32.09 | 32.11 | 32.10 |
| | | | gene | | | |
| Formula 4 | Sodium | 0.1% | O | 34.03 | 34.50 | 34.27 |
| | dodecyl | | gene | | | |
| | sulfate | | N | 35.53 | 35.71 | 35.62 |
| | | | gene | | | |
| Formula 5 | Sodium | 0.1% | O | 33.99 | 34.22 | 34.11 |
| | tetradecyl | | gene | | | |
| | sulfate | | N | 34.86 | 34.76 | 34.81 |
| | | | gene | | | |
| Formula 6 | Sodium | 0.1% | O | 34.02 | 34.32 | 34.17 |
| | hexadecyl | | gene | | | |
| | sulfate | | N | 35.34 | 34.68 | 35.01 |
| | | | gene | | | |

As can be seen from the above detection results, the anionic surfactant is optionally sodium hexadecyl sulfate, and with the same release and detection performance, the longer the hydrocarbon chain, the lower the critical micelle concentration.

### Example 3

This example provides a viral nucleic acid sample release agent comprising the following: 0.32 mmol/L NaOH, 0.01-0.05% (mass-volume ratio) sodium hexadecyl sulfate, 0-10% (mass-volume ratio) propylene glycol, 1-5% (mass-volume ratio) NP-40, 50-150 mmol/L NaCl, 1% (mass-volume ratio) EDTA, 0.075 mmol/L trehalose, and 5% (mass-volume ratio) bio-rex 70. The specific test plan is as follows in Table 7 below.

TABLE 7
Test plan

| | Sodium | Propylene | Sodium | |
|---|---|---|---|---|
| | hexadecyl sulfate | glycol | chloride | NP-40 |
| Formula 1 | 0.010% | 0% | 50 mmol/L | 1% |
| Formula 2 | 0.010% | 5% | 150 mmol/L | 3% |
| Formula 3 | 0.010% | 10% | 100 mmol/L | 5% |
| Formula 4 | 0.025% | 0% | 150 mmol/L | 5% |
| Formula 5 | 0.025% | 5% | 100 mmol/L | 1% |
| Formula 6 | 0.025% | 10% | 50 mmol/L | 3% |
| Formula 7 | 0.050% | 0% | 100 mmol/L | 3% |
| Formula 8 | 0.050% | 5% | 50 mmol/L | 5% |
| Formula 9 | 0.050% | 10% | 150 mmol/L | 1% |

The experimental method was the same as that of Example 1, and the detection results are shown in Table 8 and Table 9 below.

TABLE 8
Detection results of samples treated at room temperature

| Room | Sodium | | | | | | | |
|---|---|---|---|---|---|---|---|---|
| temperature | hexadecyl | Propylene | Sodium | | | Repeti- | Repeti- | Mean |
| treatment | sulfate | glycol | chloride | NP-40 | Target | tion 1 | tion 2 | value |
| Formula 1 | 0.010% | 0% | 50 | 1% | O gene | 33.40 | 33.70 | 33.55 |
| | | | mmol/L | | N gene | 34.99 | 34.33 | 34.66 |
| Formula 2 | 0.010% | 5% | 150 | 3% | O gene | 31.05 | 31.08 | 31.07 |
| | | | mmol/L | | N gene | 32.14 | 32.15 | 32.14 |
| Formula 3 | 0.010% | 10% | 100 | 5% | O gene | 33.21 | 33.74 | 33.48 |
| | | | mmol/L | | N gene | 34.55 | 34.69 | 34.62 |
| Formula 4 | 0.025% | 0% | 150 | 5% | O gene | 32.84 | 32.76 | 32.80 |
| | | | mmol/L | | N gene | 33.72 | 33.28 | 33.50 |
| Formula 5 | 0.025% | 5% | 100 | 1% | O gene | 30.06 | 30.02 | 30.04 |
| | | | mmol/L | | N gene | 31.11 | 31.11 | 31.11 |
| Formula 6 | 0.025% | 10% | 50 | 3% | O gene | 32.82 | 32.36 | 32.59 |
| | | | mmol/L | | N gene | 34.14 | 33.23 | 33.69 |
| Formula 7 | 0.05% | 0% | 100 | 3% | O gene | 32.27 | 32.24 | 32.25 |
| | | | mmol/L | | N gene | 33.14 | 33.18 | 33.16 |
| Formula 8 | 0.05% | 5% | 50 | 5% | O gene | 31.09 | 31.16 | 31.12 |
| | | | mmol/L | | N gene | 32.06 | 32.01 | 32.03 |
| Formula 9 | 0.05% | 10% | 150 | 1% | O gene | 33.41 | 33.55 | 33.48 |
| | | | mmol/L | | N gene | 35.01 | 34.48 | 34.74 |

TABLE 9
Detection results of samples heated

| | Sodium | | | | | | | |
|---|---|---|---|---|---|---|---|---|
| Heating | hexadecyl | Propylene | Sodium | | | Repeti- | Repeti- | Mean |
| treatment | sulfate | glycol | chloride | NP-40 | Target | tion 1 | tion 2 | value |
| Formula 1 | 0.010% | 0% | 50 | 1% | O gene | 30.05 | 30.21 | 30.13 |
| | | | mmol/L | | | | | |
| | | | | | N gene | 31.19 | 31.17 | 31.18 |
| Formula 2 | 0.010% | 5% | 150 | 3% | O gene | 30.09 | 30.13 | 30.11 |
| | | | mmol/L | | | | | |
| | | | | | N gene | 31.24 | 31.21 | 31.23 |
| Formula 3 | 0.010% | 10% | 100 | 5% | O gene | 32.22 | 31.54 | 31.88 |
| | | | mmol/L | | | | | |
| | | | | | N gene | 32.95 | 33.45 | 33.20 |
| Formula 4 | 0.025% | 0% | 150 | 5% | O gene | 29.93 | 30.07 | 30 |
| | | | mmol/L | | | | | |
| | | | | | N gene | 30.84 | 30.73 | 30.78 |
| Formula 5 | 0.025% | 5% | 100 | 1% | O gene | 29.08 | 29.06 | 29.07 |
| | | | mmol/L | | | | | |
| | | | | | N gene | 30.13 | 30.14 | 30.14 |
| Formula 6 | 0.025% | 10% | 50 | 3% | O gene | 31.37 | 31.21 | 31.29 |
| | | | mmol/L | | | | | |
| | | | | | N gene | 32.50 | 31.93 | 32.21 |
| Formula 7 | 0.05% | 0% | 100 | 3% | O gene | 30.13 | 30.2 | 30.16 |
| | | | mmol/L | | | | | |
| | | | | | N gene | 31.05 | 31.05 | 31.05 |
| Formula 8 | 0.05% | 5% | 50 | 5% | O gene | 30.13 | 30.23 | 30.18 |
| | | | mmol/L | | | | | |
| | | | | | N gene | 31.08 | 31.03 | 31.05 |
| Formula 9 | 0.05% | 10% | 150 | 1% | O gene | 32.36 | 31.54 | 31.95 |
| | | | mmol/L | | | | | |
| | | | | | N gene | 33.14 | 32.62 | 32.88 |

As can be seen from the above detection results, the sodium hexadecyl sulfate concentration is optionally 0.025%, the propylene glycol concentration is optionally 5%, the NaCl concentration is 100 mmol/L, and the NP-40 concentration is optionally 1%.

### Example 4

This example provides a viral nucleic acid sample release agent comprising the following: 0.32 mmol/L NaOH, 0.025% (mass-volume ratio) sodium hexadecyl sulfate, 5% (mass-volume ratio) propylene glycol, 1% (mass-volume ratio) NP-40, 100 mmol/L NaCl, 1% (mass-volume ratio) EDTA, 0.075 mmol/L trehalose, and 0-5% (mass-volume ratio) ion exchange resin, comprising chelex-100 (50-100 mesh), chelex-100 (100-200 mesh), chelex-100 (200-400 mesh), and bio-rex 70. The specific test plan is as follows in Table 10 below:

TABLE 10
Test plan

| | Cation exchange resin type | Concentration |
|---|---|---|
| Formula 1 | bio-rex | 0% |
| Formula 2 | bio-rex | 2.50% |
| Formula 3 | bio-rex | 5% |
| Formula 4 | chelex 100 (50-100mesh) | 2.50% |
| Formula 5 | chelex 100 (100-200mesh) | 2.50% |
| Formula 6 | chelex 100 (200-400mesh) | 2.50% |

The experimental method was the same as that of Example 1, and the detection results are shown in Table 11 and Table 12 below:

TABLE 11
Detection results of samples treated at room temperature

| Room | | | | | | |
|---|---|---|---|---|---|---|
| temperature | Ion exchange | Concen- | | Repeti- | Repeti- | Mean |
| treatment | resin type | tration | Target | tion 1 | tion 2 | value |
| Formula 1 | bio-rex | 0% | O gene | 32.46 | 32.45 | 32.45 |
| | | | N gene | 33.57 | 33.53 | 33.55 |
| Formula 2 | bio-rex | 2.50% | O gene | 30.54 | 30.41 | 30.47 |
| | | | N gene | 31.76 | 31.61 | 31.69 |
| Formula 3 | bio-rex | 5% | O gene | 30.46 | 30.39 | 30.42 |
| | | | N gene | 31.70 | 31.61 | 31.65 |
| Formula 4 | chelex 100 | 2.50% | O gene | 30.54 | 30.34 | 30.44 |
| | (50- | | | | | |
| | 100mesh) | | N gene | 31.54 | 31.69 | 31.61 |
| Formula 5 | chelex 100 | 2.50% | O gene | 30.41 | 30.41 | 30.41 |
| | (100- | | | | | |
| | 200mesh) | | N gene | 31.76 | 31.57 | 31.66 |
| Formula 6 | chelex 100 | 2.50% | O gene | 30.32 | 30.35 | 30.33 |
| | (200- | | | | | |
| | 400mesh) | | N gene | 31.79 | 31.55 | 31.67 |

TABLE 12
Detection results of samples heated

| | Cation | | | | | |
|---|---|---|---|---|---|---|
| Heating | exchange | Concen- | | Repeti- | Repeti- | Mean |
| treatment | resin type | tration | Target | tion 1 | tion 2 | value |
| Formula 1 | bio-rex | 0% | O gene | 31.65 | 31.59 | 31.62 |
| | | | N gene | 32.74 | 32.71 | 32.73 |
| Formula 2 | bio-rex | 2.50% | O gene | 29.66 | 29.51 | 29.59 |
| | | | N gene | 30.92 | 30.72 | 30.82 |
| Formula 3 | bio-rex | 5% | O gene | 29.65 | 29.57 | 29.61 |
| | | | N gene | 30.89 | 30.73 | 30.81 |
| Formula 4 | chelex 100 | 2.50% | O gene | 29.66 | 29.53 | 29.59 |
| | (50-100mesh) | | N gene | 30.72 | 30.80 | 30.76 |
| Formula 5 | chelex 100 | 2.50% | O gene | 29.55 | 29.59 | 29.57 |
| | (100-200mesh) | | N gene | 30.93 | 30.76 | 30.84 |
| Formula 6 | chelex 100 | 2.50% | O gene | 29.51 | 29.53 | 29.52 |
| | (200-400mesh) | | N gene | 30.97 | 30.71 | 30.84 |

The sample treatment and nucleic acid extraction experiments described above were repeated, and a novel coronaviral nucleic acid detection kit (isothermal amplification method) (Shanghai BioGerm Medical Technology Co., Ltd.) was used to perform detection on an isothermal nucleic acid amplification detection analyzer (BG-Nova-X8) to determine whether the sample was positive or negative. The detection results are shown in Table 13 and Table 14 below:

TABLE 13
Detection results of room temperature treatment

| Room temperature | Cation exchange | | | |
|---|---|---|---|---|
| treatment | resin type | Concentration | Repetition 1 | Repetition 2 |
| Formula 1 | bio-rex | 0% | Positive | Positive |
| Formula 2 | bio-rex | 2.50% | Positive | Positive |
| Formula 3 | bio-rex | 5% | Positive | Positive |
| Formula 4 | chelex 100 (50-100mesh) | 2.50% | Positive | Positive |
| Formula 5 | chelex 100 (100-200mesh) | 2.50% | Positive | Positive |
| Formula 6 | chelex 100 (200-400mesh) | 2.50% | Positive | Positive |

TABLE 14
Detection results of heating treatment

| Heating | Cation exchange | Con- | | |
|---|---|---|---|---|
| treatment | resin type | centration | Repetition 1 | Repetition 2 |
| Formula 1 | bio-rex | 0% | Positive | Positive |
| Formula 2 | bio-rex | 2.50% | Positive | Positive |
| Formula 3 | bio-rex | 5% | Positive | Positive |
| Formula 4 | chelex 100 | 2.50% | Positive | Positive |
| | (50-100mesh) | | | |
| Formula 5 | chelex 100 | 2.50% | Positive | Positive |
| | (100-200mesh) | | | |
| Formula 6 | chelex 100 | 2.50% | Positive | Positive |
| | (200-400mesh) | | | |

As can be seen from the above detection results, the ion exchange resin is optionally bio-rex70, and the concentration is optionally 2.5%.

### Example 5

This example provides a viral nucleic acid sample release agent having the following composition in Table 15 below:

TABLE 15

| Composition of viral nucleic acid sample release agent | | |
|---|---|---|
| Component | Concentration | |
| NaOH | 3.2 | mmol/L |
| Sodium hexadecyl sulfate | 0.025 | % |
| Propylene glycol | 5 | % |
| NP-40 | 1 | % |
| NaCl | 100 | mmol/L |
| 0.5 M EDTA | 1 | % (v/v) |
| Bio-rex 70 | 2.5 | % (w/v) |
| Trehalose | 0.075 | mmol/L |

By using the viral nucleic acid sample release agent provided in this example, a pseudovirus sample was treated according to the experimental method provided in Example 1, and the extracted sample was separately detected with qPCR detection reagent and RAA detection reagent.

### Comparative Example 1

The viral nucleic acid release agent provided by this comparative example comprises 25 mmol/L NaOH, 1.0% Triton X 100, 2 mmol/L EDTA, and 10 mmol/L Tris HCl, and the method of use is: 20 μL of a pseudovirus sample was taken, 20 μL of the viral nucleic acid release agent was added, and the mixture was uniformly mixed by vortex, left to stand at room temperature for 1 min, and taken for use.

### Comparative Example 2

The method of use of the commercially available viral nucleic acid sample release agent product 1 (Weifang Amplification Future Biotechnology Co., Ltd., Cat. No. WLDR8202-S) is: 20 μL of a pseudovirus sample was taken, 5 μL of the viral nucleic acid sample release agent product 1 was then added, and the mixture was gently mixed and placed in a metal bath, and incubated at 95° C. for 5 min; the extracted sample was taken out, equilibrated at room temperature for 3 min, and centrifuged at 10000 rpm for 2 min; the supernatant was taken to directly carry out the subsequent reaction.

### Comparative Example 3

DEPC water was used as a viral nucleic acid sample release agent, 20 μL of a pseudovirus sample was taken, 20 μL of the viral nucleic acid sample release agent was added, and the mixture was uniformly mixed by vortex, left to stand at room temperature for 5 min and subjected to subsequent detection.

### Comparative Example 4

The viral nucleic acid sample release agent in this comparative example consists of 50 mmol/L guanidinium isothiocyanate, 0.05% (by volume) Tween-20, 0.05% Triton X-100, 25% ethanol, 20% isoamyl alcohol, and sterile enzyme-free water. The method of use is that the viral nucleic acid sample release agent was mixed with the virus sample at the ratio of 1:1, and the mixture was left to stand for 30 min for subsequent detection.

### Comparative Example 5

Commercially available magnetic bead nucleic acid extraction reagent from Guangzhou Magen Biotechnology Co., Ltd., Guangdong Guangzhou Medical Device Filing No. 20150062.

### Comparative Example 6

The viral nucleic acid sample release agent in this comparative example comprises 0.1 M (molar concentration) NaOH, 1% (volume percentage (mL/mL)) NP40 (ethylphenylpolyethylene glycol), 0.5% (mass-volume percentage (mg/mL)) LLS, 0.1 M (molar concentration) guanidinium isothiocyanate, 0.2% (mass-volume percentage) SDS, and 0.5% (mass-volume percentage) Foam ban.

The extraction effects of Example 5 and Comparative Examples 1-6 described above were detected, and the experimental method is:

### (1) Sample Preparation:

The pseudovirus of novel coronavirus was taken at an initial concentration of 106 copies/mL and diluted to 105 copies/mL for later use using the sampling set from Youkanghengye after the negative swab was collected.

### (2) Sample Treatment and Nucleic Acid Extraction:

The prepared pseudovirus samples were separately treated at room temperature and under a heated condition using the viral nucleic acid sample release agent in Example 5 of the present disclosure, and sample treatment was also separately performed using the viral nucleic acid sample release agents provided in Comparative Example 1, Comparative Example 2, Comparative Example 3, Comparative Example 4, and Comparative Example 6. 200 μL of the sample was taken for extraction using Comparative Example 5. The extracted nucleic acids were detected using a commercially available novel coronaviral nucleic acid detection kit (fluorescence PCR method) (Shanghai BioGerm Medical Technology Co., Ltd.) and a novel coronaviral nucleic acid detection kit (fluorescence PCR method) (Guangzhou Daan Gene Co., Ltd.).

The sample treatment and nucleic acid extraction experiments described above were repeated, and a novel coronaviral nucleic acid detection kit (isothermal amplification method) (Shanghai BioGerm Medical Technology Co., Ltd.) was used to perform detection on an isothermal nucleic acid amplification detection analyzer (BG-Nova-X8) to determine whether the sample was positive or negative.

The detection results of the novel coronaviral nucleic acid detection kit (fluorescence PCR method) (Shanghai BioGerm Medical Technology Co., Ltd.) are shown in Table 16 below:

TABLE 16
Comparison of detection results

| | | Repetition | Repetition | Mean |
|---|---|---|---|---|
| | Target | 1 | 2 | value |
| Example 5-room | O gene | 30.42 | 30.99 | 30.71 |
| temperature | N gene | 30.91 | 31.24 | 31.08 |
| Example 5-heating | O gene | 29.05 | 29.1 | 29.08 |
| | N gene | 29.31 | 29.88 | 29.59 |
| Comparative Example 1 | O gene | 33.82 | 34.39 | 34.11 |
| | N gene | 33.85 | 35.02 | 34.44 |
| Comparative Example 2 | O gene | 33.63 | 34.68 | 34.16 |
| | N gene | 34.21 | 34.87 | 34.54 |
| Comparative Example 3 | O gene | 33.82 | 34.39 | 34.11 |
| | N gene | 33.93 | 34.87 | 34.40 |
| Comparative Example 4 | O gene | 37.57 | 35.24 | 36.41 |
| | N gene | 38.37 | 35.96 | 37.16 |
| Comparative Example 5 | O gene | 31.38 | 31.66 | 31.52 |
| | N gene | 31.66 | 31.97 | 31.81 |
| Comparative Example 6 | O gene | 32.36 | 32.6 | 32.48 |
| | N gene | 33.00 | 33.06 | 33.03 |

The detection results of the novel coronaviral nucleic acid detection kit (fluorescence PCR method) (Guangzhou Daan Gene Co., Ltd.) are shown in Table 17 below:

TABLE 17
Comparison of detection results

| | | Repetition | Repetition | Mean |
|---|---|---|---|---|
| | Target | 1 | 2 | value |
| Example 5-room | O gene | 20.41 | 20.94 | 20.67 |
| temperature | N gene | 21.09 | 21.51 | 21.30 |
| Example 5-heating | O gene | 19.01 | 19.01 | 19.01 |
| | N gene | 19.55 | 19.59 | 19.57 |
| Comparative Example 1 | O gene | 23.75 | 24.37 | 24.06 |
| | N gene | 24.13 | 24.45 | 24.29 |
| Comparative Example 2 | O gene | 23.57 | 24.68 | 24.12 |
| | N gene | 23.75 | 24.88 | 24.32 |
| Comparative Example 3 | O gene | 23.81 | 24.29 | 24.05 |
| | N gene | 24.26 | 24.39 | 24.32 |
| Comparative Example 4 | O gene | 27.57 | 25.15 | 26.36 |
| | N gene | 27.87 | 25.18 | 26.52 |
| Comparative Example 5 | O gene | 21.34 | 21.63 | 21.48 |
| | N gene | 21.68 | 21.87 | 21.78 |
| Comparative Example 6 | O gene | 22.32 | 22.51 | 21.48 |
| | N gene | 22.89 | 22.96 | 22.93 |

The detection results of the novel coronaviral nucleic acid detection kit (isothermal amplification method) (Shanghai BioGerm Medical Technology Co., Ltd.) are shown in Table 18 below:

TABLE 18
Comparison of detection results

| | | Repetition 1 | Repetition 2 |
|---|---|---|---|
| Example 5-room temperature | O gene | Positive | Positive |
| | N gene | Positive | Positive |
| Example 5-heating | O gene | Positive | Positive |
| | N gene | Positive | Positive |
| Comparative Example 1 | O gene | Negative | Negative |
| | N gene | Negative | Negative |
| Comparative Example 2 | O gene | Negative | Negative |
| | N gene | Negative | Negative |
| Comparative Example 3 | O gene | Negative | Negative |
| | N gene | Negative | Negative |
| Comparative Example 4 | O gene | Positive | Negative |
| | N gene | Positive | Positive |
| Comparative Example 5 | O gene | Positive | Positive |
| | N gene | Positive | Positive |
| Comparative Example 5 | O gene | Negative | Negative |
| | N gene | Negative | Negative |
| Comparative Example 6 | O gene | Negative | Negative |
| | N gene | Negative | Negative |

According to the above experimental results, the viral nucleic acid sample release agent provided by the present disclosure can obtain a relatively good detection results in qPCR detection reagents of different manufacturers. Also, in the detection of isothermal RAA, a relatively good experimental result can be obtained.

### Example 6

Commercially available non-inactivated sampling sets from three manufacturers were selected, the viral nucleic acid sample release agent provided in Example 5 was used to separately treat the pseudovirus samples diluted by the three different sampling sets under room temperature treatment and heating treatment, and then qPCR detection reagent and RAA detection reagent were separately used for detection. The experimental procedures are as follows:

### (1) Sample Preparation:

The pseudovirus of novel coronavirus was taken at an initial concentration of 106 copies/mL and diluted to 105 copies/mL for later use using the three non-inactivated sampling sets after the negative swab was collected.

### (2) Sample Treatment and Nucleic Acid Extraction:

The prepared pseudovirus samples were separately treated at room temperature and under a heated condition using the sample release agent of the present disclosure. The extracted nucleic acids were detected using a commercially available novel coronaviral nucleic acid detection kit (fluorescence PCR method) (Shanghai BioGerm Medical Technology Co., Ltd.).

The sample treatment and nucleic acid extraction experiments described above were repeated, and a novel coronaviral nucleic acid detection kit (isothermal amplification method) (Shanghai BioGerm Medical Technology Co., Ltd.) was used to perform detection on an isothermal nucleic acid amplification detection analyzer (BG-Nova-X8) to determine whether the sample was positive or negative.

The detection results of the novel coronaviral nucleic acid detection kit (fluorescence PCR method) (Shanghai BioGerm Medical Technology Co., Ltd.) are shown in Table 19 and Table 20 below:

TABLE 19
Detection results of room temperature treatment

| Room temperature treatment | Target | Repetition 1 | Repetition 2 | AVG |
|---|---|---|---|---|
| Manufacturer 1 | O gene | 30.10 | 30.34 | 30.22 |
| | N gene | 31.02 | 31.11 | 31.07 |
| Manufacturer 2 | O gene | 30.46 | 30.61 | 30.53 |
| | N gene | 32.06 | 31.82 | 31.94 |
| Manufacturer 3 | O gene | 29.93 | 30.56 | 30.25 |
| | N gene | 31.83 | 31.60 | 31.71 |

TABLE 20
Detection results of heating treatment

| Heating treatment | Target | Repetition 1 | Repetition 2 | AVG |
|---|---|---|---|---|
| Manufacturer 1 | O gene | 29.11 | 29.4 | 29.26 |
| | N gene | 30.05 | 30.14 | 30.10 |
| Manufacturer 2 | O gene | 29.55 | 29.76 | 29.66 |
| | N gene | 31.06 | 30.98 | 31.02 |
| Manufacturer 3 | O gene | 29.04 | 29.57 | 29.31 |
| | N gene | 30.98 | 30.67 | 30.83 |

The detection results of the novel coronaviral nucleic acid detection kit (isothermal amplification method) (Shanghai BioGerm Medical Technology Co., Ltd.) are shown in Table 21 below:

TABLE 21
Detection results

| | Repetition 1 | Repetition 2 |
|---|---|---|
| Manufacturer 1 | Positive | Positive |
| Manufacturer 2 | Positive | Positive |
| Manufacturer 3 | Positive | Positive |

From the above experimental results, it can be seen that the viral nucleic acid sample release agent provided by the present disclosure can be adapted to qPCR and RAA detection in sampling sets of three manufacturers.

Finally, it should be noted that the above examples are only used to illustrate the technical solution of the present disclosure, and should not limit the same. Although the present disclosure is described in detail with reference to the examples described above, it will be appreciated by those skilled in the art that, the technical solution in the examples described above can still be modified, or some or all of the technical features can be equivalently substituted. Such modifications or substitutions do not make the technical solution corresponding thereto depart from the scope of the technical solution in the examples of the present disclosure.

### INDUSTRIAL APPLICABILITY

The viral nucleic acid sample release agent provided by the present disclosure can be compatible with various sampling sets, and by adjusting the types and the proportions of the components, the critical micelle concentration of the used surfactant is reduced to the maximum extent, so that the lysis on cells and pathogens can be ensured, and the inhibition on the subsequent detection system can also be reduced. According to the present disclosure, when an ion exchange resin is added, proteins, metal ions such as calcium and magnesium which have influences on subsequent reactions, and the like in a sample can be adsorbed, cell and virus lysis can be promoted, and the compatibility of the system is enhanced. The detection system of the present disclosure has relatively good compatibility, can be compatible with various qPCR detection systems, and can also be used for an isothermal RAA amplification system. The present disclosure is not only suitable for a liquid detection system, but also can be used for a dry powder detection system with a large sample size. Therefore, the viral nucleic acid sample diluent, the kit for extracting viral nucleic acid from samples, and the method for extracting viral nucleic acid from samples provided by the present disclosure have excellent utility.

What is claimed is:

1. A viral nucleic acid sample diluent, comprising: an anionic surfactant, sodium hydroxide, EDTA, trehalose, and an ion exchange resin, wherein an addition amount of the EDTA is 0-3% according to mass-volume ratio; an addition amount of the anionic surfactant is 0.010%-0.050%; and an addition amount of the ion exchange resin is 0-5%.

2. The viral nucleic acid sample diluent according to claim 1, wherein the anionic surfactant is selected from sodium dodecyl sulfate, sodium tetradecyl sulfate, sodium hexadecyl sulfate, and sodium octadecyl sulfate.

3. The viral nucleic acid sample diluent according to claim 1, wherein a concentration of the sodium hydroxide is 0.16-1.28 mmol/L.

4. The viral nucleic acid sample diluent according to claim 1, further comprising one or a combination of two or more of polyol, sodium chloride, or NP-40.

5. The viral nucleic acid sample diluent according to claim 4, wherein the polyol is selected from ethylene glycol and propylene glycol:
wherein the polyol is 0-10% propylene glycol according to mass-volume ratio;
wherein a concentration of the NP-40 is 1%-5% according to mass-volume ratio;
wherein a concentration of the sodium chloride is 50-150 mmol/L.

6. The viral nucleic acid sample diluent according to claim 1, wherein the ion exchange resin is selected from chelex and bio-rex 70, and the chelex has a specification of 50-400 mesh.

7. The viral nucleic acid sample diluent according to claim 1, wherein a concentration of the trehalose is 0.05-0.1 mmol/L.

8. A kit for extracting viral nucleic acid from samples, comprising the viral nucleic acid sample diluent according to claim 1.

9. A method for extracting viral nucleic acid from samples, comprising: uniformly mixing a virus sample with the viral nucleic acid sample diluent according to claim 1, and obtaining the viral nucleic acid after completing the reaction.

10. The method for extracting viral nucleic acid from samples according to claim 9, wherein the method for extracting viral nucleic acid from samples comprises the following steps: uniformly mixing the virus sample with the viral nucleic acid sample diluent, and then heating, and obtaining the viral nucleic acid after completing the reaction:
wherein the heating is performed at a temperature of 90-110° C., with a period of 1-5 min.

11.-12. (canceled)

13. A method for diagnosing a disease associated with a virus infection in a subject, comprising:
A) uniformly mixing the viral nucleic acid sample diluent according to claim 1 with a virus sample from the subject, obtaining a viral nucleic acid after completing the reaction, and performing a detection via a virus detection reagent; and
B) determining the source of the virus.

14. The method according to claim 13, wherein the virus comprises a novel coronavirus.

15. The viral nucleic acid sample diluent according to claim 1, wherein an addition amount of the EDTA is 1%; an addition amount of the anionic surfactant is 0.02%; and an addition amount of the ion exchange resin is 2.5%.

16. The viral nucleic acid sample diluent according to claim 1, wherein the anionic surfactant is selected from sodium hexadecyl sulfate.

17. The viral nucleic acid sample diluent according to claim 1, wherein a concentration of the sodium hydroxide is 0.32 mmol/L.

18. The viral nucleic acid sample diluent according to claim 5, wherein the polyol is 5% propylene glycol according to mass-volume ratio:
wherein a concentration of the NP-40 is 1% according to mass-volume ratio;
wherein a concentration of the sodium chloride is 100 mmol/L.

19. The viral nucleic acid sample diluent according to claim 1, wherein the ion exchange resin is bio-rex 70.

20. The viral nucleic acid sample diluent according to claim 1, wherein a concentration of the trehalose is 0.075 mmol/L.

21. The kit for extracting viral nucleic acid from samples according to claim 8, wherein the viral nucleic acid sample extraction kit comprises a heating device.

22. The method for extracting viral nucleic acid from samples according to claim 9, wherein the reaction is performed for a period of 5-15 min.
